(19) United States
(12) Patent Application Publication
(10) Pub. No.: US 2006/0046286 A1
(43) Pub. Date: Mar. 2, 2006

(54) Method for preparing optically active beta-butyrolactones

(76) Inventors: Shinya Watanabe, Kanagawa (JP); Mitsuhiko Fujiwhara, Tokyo (JP); Noboru Sayo, Tokyo (JP)

Correspondence Address: WENDEROTH, LIND & PONACK, L.L.P., 2033 K STREET N. W., SUITE 800, WASHINGTON, DC, 20006-1021, US

(21) Appl. No.: 11/209,643

(22) Filed: Aug. 24, 2005

(30) Foreign Application Priority Data
Aug. 25, 2004 (JP) 2004-245656

(51) Int. Cl. C12P 17/04; C12P 41/00

(52) U.S. Cl. 435/126; 435/280; 435/74; 549/313

(57) ABSTRACT

The present invention is to provide a method for preparing easily and efficiently β-butyrolactones and/or optically active 3-hydroxycarboxylic acid derivatives in high optical purity by use of an easily available hydrolase, which are useful as intermediates for pharmaceuticals, agrochemicals and the like. More particularly, the present invention relates to a method for preparing β-butyrolactones and/or optically active 3-hydroxycarboxylic acid derivatives with an optical purity of substantially 100% ee, comprising reacting a β-butyrolactone which is a mixture of optical isomers, with a nucleophilic agent in the presence of a hydrolase, provided that a lipase derived from porcine pancreas is excluded.

### BACKGROUND OF THE INVENTION

1. Field of the Invention

The present invention relates to a method for preparing optically active β-butyrolactones which are useful as intermediates for pharmaceuticals, agrochemicals, and the like.

2. Description of the Related Art

It is known that unique reactions different from chemical techniques occur in reactions using enzymes or microorganisms, because such enzymes or microorganisms themselves have properties such as specific selectivity, and the like. As reactions like these, there have been reported optical resolution of racemates, and highly selective isomerization from one of optical isomers (mirror image) which is a mixture of geometric isomers, into another isomer under mild conditions.

On the other hand, various methods for preparing optically active β-butyrolactones have been reported. As the reactions using enzymes or microorganisms as mentioned above, for example, there has been described a method that optically active (R)-β-butyrolactone is produced by treating a racemic β-butyrolactone with a porcine pancreas lipase in the presence of benzyl alcohol [J. Chem. Soc., Perkin Trans. 1, 1645-1646 (1995) and J. Chem. Soc., Perkin Trans. 1, 71-77 (2000)]. However, since the lipase derived from porcine pancreas is an animal-derived enzyme, such known methods described in said literatures have problems that it is difficult to supply a large amount of such lipase, and it is necessary to use the enzyme in an amount equal to that of the substrate. Moreover, such known methods are not an industrial production method, because they require 6 days to perform the reaction.

### SUMMARY OF THE INVENTION

The present invention has been made under those circumstances as mentioned above, and easily available hydrolases can be used in the present invention. Thus, an object of the present invention is to provide a method for preparing easily and efficiently P-butyrolactones and/or 3-hydroxycarboxylic acid derivatives having high optical purity.

In order to solve the above-mentioned problems, the present inventors have studied intensively, and as a result, they have found that optically active β-butyrolactones and/or 3-hydroxycarboxylic acid derivatives can be prepared by reacting a β-butyrolactone which is a mixture of optical isomers, with a nucleophilic agent in the presence of a hydrolase, provided that a lipase derived from porcine pancreas is excluded, and in particular, when a lipase derived from the genus Candida, (R)-β-butyrolactone and/or (S)-3-hydroxycarboxylic acid derivative can be prepared in high optical purity. The present invention has been completed, based on these findings.

That is, the present invention relates to the followings:
(1) a method for preparing an optically active β-butyrolactone, which comprises reacting a β-butyrolactone which is a mixture of optical isomers, with a nucleophilic agent in the presence of a hydrolase, provided that a lipase derived from porcine pancreas is excluded,
(2) the method according to the above (1), wherein the optically active β-butyrolactone obtained is (R)-β-butyrolactone,
(3) the method according to the above (1), wherein the β-butyrolactone which is a mixture of optical isomers is a β-butyrolactone with low optical purity, and the β-butyrolactone obtained is a β-butyrolactone with high optical purity,
(4) the method according to the above (1), wherein the optical purity of the β-butyrolactone obtained is substantially 100% ee,
(5) a method for preparing an optically active β-butyrolactone and/or an optically active 3-hydroxycarboxylic acid derivative, which comprises reacting a β-butyrolactone which is a mixture of optical isomers, with a nucleophilic agent in the presence of a hydrolase, provided that a lipase derived from porcine pancreas is excluded,
(6) the method according to the above (5), wherein the optically active β-butyrolactone obtained and/or the optically active 3-hydroxycarboxylic acid derivative obtained is/are isolated,
(7) the method according to the above (5) or (6), wherein the 3-hydroxycarboxylic acid derivative obtained is an optically active 3-hydroxycarboxylic acid derivative,
(8) the method according to any one of the above (5) or (6), wherein the optically active β-butyrolactone obtained is an (R)-β-butyrolactone, and the optically active 3-hydroxycarboxylic acid derivative obtained is an (S)-3-hydroxycarboxylic acid derivative,
(9) the method according to the above (5) or (6), wherein the β-butyrolactone which is a mixture of optical isomers is a β-butyrolactone with low optical purity, and the β-butyrolactone obtained is a β-butyrolactone with high optical purity,
(10) the method according to the above (5) or (6), wherein the optical purity of the (R)-β-butyrolactone obtained is substantially 100% ee,
(11) the method according to the above (1) or (5), wherein the hydrolase is a lipase, provided that a lipase derived from porcine pancreas is excluded,
(12) the method according to the above (11), wherein the lipase is a lipase belonging to the genus Candida,
(13) the method according to the above (12), wherein the lipase belonging to the genus Candida is a lipase derived from Candida antarctica, and
(14) the method according to the above (1) or (5), wherein the nucleophilic agent is an alcohol or an amine.
(15) the method according to the above (14), wherein the alcohol is an alcohol represented by the formula (4):
   R¹-A¹-OH (4)
   wherein R¹ is an optionally substituted hydrocarbon group or a halogen atom, and A¹ is a spacer or a direct link, and the amine is an amine represented by the formula (5):
   R²-A²-NH-A²-R³ (5)
   wherein R² and R³ are each independently an optionally substituted hydrocarbon group or a halogen atom, A² and A³ are each independently a spacer or a direct link, and
(16) the method according to the above (7), wherein the optically active 3-hydroxycarboxylic acid derivative is an optically active 3-hydroxycarboyxlic acid derivative represented by the formula (3): 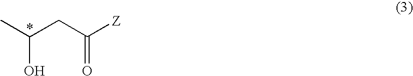
   wherein Z is a group derived from a nucleophilic agent, and the symbol * is an asymmetric carbon atom, and
(17) the method according to the above (1), wherein the optically active β-butyrolactone obtained is isolated.

### DETAILED DESCRIPTION OF THE PREFERRED EMBODIMENTS

According to the present invention, since the objective optically active β-butyrolactones with high optical purity and/or the objective optically active 3-hydroxycarboxylic acid derivative with high optical purity can be obtained by using a hydrolase other than a lipase derived from porcine pancreas under mild conditions, the present invention has excellent effects such as less production cost, no necessity for the removal of by-products and impurities, improvement in workability, and usability of commercially and easily available hydrolases, and the present invention enables more efficient production in comparison with known chemical methods. Moreover, in the case where an optically active (-butyrolactones are required, not only materials more than half become useless in the optical resolution of racemates, but also high optical purity is not necessarily attained by asymmetric hydrogenation which has been remarkably developed in recent years. Thus, according to the present invention, there is also an advantage that very much optimized optically active (-butyrolactones can be obtained in high optical purity in view of atom economy and optical purity.

Also, since the method of the present invention uses a hydrolase other than a lipase derived from porcine pancreas, the desired optically active (-butyrolactones and/or the desired optically active 3-hydroxycarboxylic acid derivatives can be obtained under mild conditions. In particular, when using an alcohol as a nucelophilic agent, (R)-(-butyrolactone and/or (S)-3-hydroxycarboxylic acid derivative is/are obtained in high optical purity of substantially 100% ee. This is also an effect of the present invention.

The β-butyrolactone, which is a mixture of optical isomers, used in the present invention is a mixture of optical isomers of β-butyrolactones represented by the formula (1): 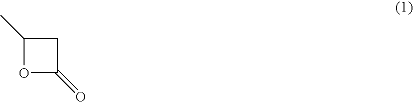
and is a mixture of (R)-β-butyrolactone represented by the formula (2A): 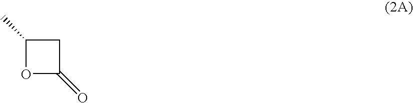
and (S)-β-butyrolactone represented by the formula (2B):

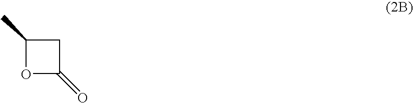

The above β-butyrolactone which is a mixture of optical isomers may be commercially available, or optically active β-butyrolactones prepared appropriately by asymmetric synthesis using, for example, asymmetric metal complexes may be employed.

The optical purity of a mixture optical isomer of β-butyrolactone, i.e., a mixture of (R)-β-butyrolactone and (S)-β-butyrolactone may be zero (i.e. the mixture is racemates) or a low optical purity. Here, “low optical purity” means an optical purity other than 100% ee, and means an optical purity of the optical isomers lower than that of the objective optically active β-butyrolactone when compared. When the β-butyrolactone which is a mixture of optical isomers has a low optical purity, such optical purity is not less than 1% ee, preferably 1 to 99% ee, more preferably 1 to 80% ee. Also, the β-butyrolactones having a high optical purity can be prepared by performing the process of the present invention even using a β-butyrolactone of which optical purity is 80 to 99% ee, preferably 90 to 95% ee.

The above optically active β-butyrolactone obtained by the method of the present invention can be an optically active β-butyrolactone having a high optical purity. Here, “a high optical purity” means an optical purity of a β-butyrolactone obtained higher than that of a β-butyrolactone which is a mixture of optical isomers when compared to such mixture of optical isomers, and means substantially 100% ee. Here, “substantially 100% ee” means an optical purity where one mirror image over another mirror image is almost not detectable. In the present invention, optical purity of substantially 100% ee can be not less than 95% ee, preferably not less than 97% ee, more preferably 98% ee, especially preferably 99% ee.

The optically active β-butyrolactone obtained by the method of the present invention can be represented by the formula (2): 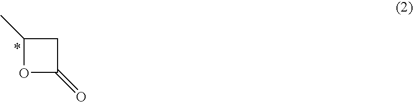
wherein * is an asymmetric carbon atom.

The optically active β-butyrolactone of the above formula (2) includes (R)-β-butyrolactone represented by the above formula (2A) and (S)-β-butyrolactone represented by the above formula (2B). In the present invention, (R)-β-butyrolactone represented by the above formula (2A) is preferable, though it depends on the kind of hydrolases to be used.

The 3-hydroxycarboxylic acid derivatives prepared according to the method of the present invention can be represented, for example, by the formula (3C): 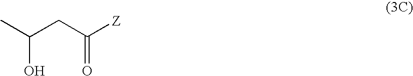
wherein Z is a group derived from a nucleophilic agent. Also, the optically active 3-hydroxycarboxylic acid derivatives of the present invention can be represented, for example, by the formula (3): 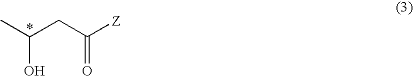
wherein Z and the symbol * are the same meanings as defined above.

In the formulae (3) and (3C), the group derived from a nucleophilic agent represented by Z includes, for example, —O-A¹-R¹ and 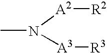
wherein R¹ is an optionally substituted hydrocarbon group or a halogen atom; R² and R³ are each independently a hydrogen atom, an optionally substituted hydrocarbon group or a halogen atom; and A¹, A² and A³ are each independently a spacer or a direct link, provided that when R¹ is a halogen atom, then A¹ is a spacer, or when R² and/or R³ is/are a halogen atom, then A and/or A³ are each a spacer; and R² and R³ may be taken together to form a ring.

Here, when the method of the present invention is performed using an alcohol as the nucelophilic agent (hereinafter described), the optically active 3-hydroxycarboxylic acid derivative represented by the above formula (3) can be, for example, an optically active 3-hydroxycarboxylic acid ester represented by the formula (3-1): 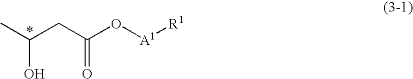
wherein R¹, A¹ and the symbol * have the same meanings as defined above, and when the method of the present invention is performed using an amine as the nucleophilic agent, the optically active 3-hydroxycarboxylic acid derivative represented by the above formula (3) can be, for example, an optically active 3-hydroxycarboxylic acid amide represented by the formula (3-2): 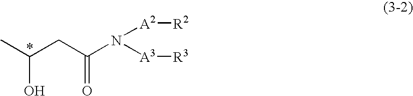
wherein R², R³, A², A³ and * have the same meanings as defined above.

In each formulae mentioned above, the halogen atom represented by R¹, R² and R³ include fluorine, chlorine, bromine, and iodine.

The optionally substituted hydrocarbon group includes a hydrocarbon group and a substituted hydrocarbon group.

The hydrocarbon group includes, for example, an alkyl group, an aryl group and an aralkyl group.

The alkyl group may be linear, branched, or cyclic, and includes, for example, an alkyl group of 1 to 20 carbon atoms, such as methyl, ethyl, n-propyl, 2-propyl, n-butyl, 2-butyl, isobutyl, tert-butyl, n-pentyl, 2-pentyl, tert-pentyl, 2-methylbutyl, 3-methylbutyl, 2,2-dimethylpropyl, n-hexyl, 2-hexyl, 3-hexyl, tert-hexyl, 2-methylpentyl, 3-methylpentyl, 4-methylpentyl, heptyl, octyl, nonyl, decyl, lauryl, stearyl, cyclopropyl, cyclobutyl, cyclopentyl, cyclohexyl, and the like, among which an alkyl group of 1 to 11 carbon atoms is preferable.

Example of the aryl group is an aryl group having 6 to 20 carbon atom, including specifically phenyl, naphthyl, anthryl, biphenyl, tolyl, xylyl, and the like, among which an aryl group of 6 to 10 carbon atoms is preferable.

The aralkyl group can be a group formed by substituting at least one hydrogen atom of the above alkyl group, with the above aryl group, such as aralkyl group having 7 to 20 carbon atoms, including specifically benzyl, 2-phenylethyl, 1-phenylpropyl, 3-naphthylpropyl and the like, among which the aralkyl group of 7 to 12 carbon atoms is preferable.

The substituted hydrocarbon group (hydrocarbon groups having substituent(s)) can be a hydrocarbon group formed by substituting at least one hydrogen atom of the above hydrocarbon group, with a substituent. Examples of such substituted hydrocarbon groups are a substituted alkyl group, a substituted aryl group, a substituted aralkyl group, and the like.

The substituent includes, for example, an optionally substituted hydrocarbon group, a halogen atom, a halogenated hydrocarbon group, an optionally substituted heterocyclic group, an optionally substituted alkoxy group, an optionally substituted aryloxy group, an optionally substituted aralkyloxy group, an optionally substituted heteroaryloxy group, an optionally substituted alkylthio group, an optionally substituted arylthio group, an optionally substituted aralkylthio group, an optionally substituted heteroarylthio group, an optionally substituted acyl group, an optionally substituted acyloxy group, an optionally substituted alkoxycarbonyl group, an optionally substituted aryloxycarbonyl group, an optionally substituted aralkyloxycarbonyl group, an optionally substituted alkylenedioxy group, a nitro group, an amino group, a substituted amino group, a cyano group, a sulfo group, a substituted silyl group, a hydroxy group, a carboxy group, an optionally substituted alkoxythiocarbonyl group, an optionally substituted aryloxythiocarbonyl group, an optionally substituted aralkyloxythiocarbonyl group, an optionally substituted alkylthiocarbonyl group, an optionally substituted arylthiocarbonyl group, an optionally substituted aralkylthiocarbonyl group, an optionally substituted carbamoyl group, a substituted phosphino group, an aminosulfonyl group, an alkoxysulfonyl group, and the like.

The optionally substituted hydrocarbon group and halogen atom which are a substituent are each the same as those mentioned above.

Examples of the spacer represented by A¹ to A³ are an alkylene group, a divalent organic group (e.g. a divalent aromatic group), —[(CH₂)ₙ₁—CO—(CH₂)ₙ₂]ₙ₃—, —[(CH₂)ₙ₄—CONH—(CH₂)ₙ₅]ₙ₆—, —[(CH₂)ₙ₇—O—(CH₂)ₙ₈]ₙ₉—, and the like.

The alkylene group may be linear, branched, or cyclic, and includes, for example, an alkylene group of 1 to 6 carbon atoms, such as methylene, ethylene, propylene, butylene, 2-methylpropylene, pentylene, 2,2-dimethylpropylene, 2-ethylpropylene, hexylene, cyclohexylene, and the like.

The divalent aromatic group includes, for example, an arylene group having 6 to 12 carbon atoms, and specific examples of such arylene group are phenylene, biphenyldiyl, —CH₂C₆H₅—CH₂C₆H₄CH₂—, and the like.

Above n1 to n9 are each natural number, preferably 1 to 6, and more preferablyl 1 to 3. Further, each of —(CH₂)ₙ₁—, —(CH₂)ₙ₂—, —(CH₂)ₙ₄—, —(CH₂)ₙ₅—, —(CH₂)ₙ₇— and —(CH₂)ₙ₈— may be a linear or branched chain. That is, the unit of —(CH₂)— is not a mere repetition unit, but a conventional unit for representing the number of carbon atoms and hydrogen atoms. Accordingly, for example, in the case where —(CH₂)ₙ₁— is a branched chain and n1 is 3, such —(CH₂)ₙ₁— can be —CH(CH₃)—CH₂—, —CH₂—CH(CH₃)—, —CH(CH₂CH₃)— or —C(CH₃)₂—. When the alkylene group is 2, it becomes to be —CH(CH₃)—.

When R² and R³, taken together, form a ring, such ring may be a monocyclic ring, a polycyclic ring, or a fused ring. For example, 4- to 8-membered aliphatic rings are included. Further, the ring system may contain —O—, —NH—, —S—, carbonyl (C═O), thiocarbonyl (C═S), and the like in the ring-constituent carbon chain. Examples of the ring when formed include cyclopentane ring, cyclohexane ring, for example 5- to 7-membered lactone ring, and for example 5- to 7-membered lactam ring.

Examples of the optically active 3-hydroxycarboxylic acid esters represented by the above formula (3-1) include, for example,
methyl 3-hydroxybutanoate,
ethyl 3-hydroxybutanoate,
n-propyl 3-hydroxybutanoate,
2-propyl 3-hydroxybutanoate,
n-butyl 3-hydroxybutanoate,
2-butyl 3-hydroxybutanoate,
isobutyl 3-hydroxybutanoate,
tert-butyl 3-hydroxybutanoate,
n-pentyl 3-hydroxybutanoate,
2-pentyl 3-hydroxybutanoate,
tert-pentyl 3-hydroxybutanoate,
2-methylbutyl 3-hydroxybutanoate,
3-methylbutyl 3-hydroxybutanoate,
2,2-dimethylpropyl 3-hydroxybutanoate,
n-hexyl 3-hydroxybutanoate,
2-hexyl 3-hydroxybutanoate,
3-hexyl 3-hydroxybutanoate,
tert-hexyl 3-hydroxybutanoate,
2-methylpentyl 3-hydroxybutanoate,
3-methylpentyl 3-hydroxybutanoate,
4-methylpentyl 3-hydroxybutanoate,
heptyl 3-hydroxybutanoate,
octyl 3-hydroxybutanoate,
nonyl 3-hydroxybutanoate,
decyl 3-hydroxybutanoate,
lauryl 3-hydroxybutanoate,
stearyl 3-hydroxybutanoate,
cyclopropyl 3-hydroxybutanoate,
cyclobutyl 3-hydroxybutanoate,
cyclopentyl 3-hydroxybutanoate,
cyclohexyl 3-hydroxybutanoate,
phenyl 3-hydroxybutanoate,
naphthyl 3-hydroxybutanoate,
anthryl 3-hydroxybutanoate,
biphenyl 3-hydroxybutanoate,
benzyl 3-hydroxybutanoate,
2-phenylethyl 3-hydroxybutanoate,
1-phenylpropyl 3-hydroxybutanoate,
3-naphthylpropyl 3-hydroxybutanoate, etc.

The optically active 3-hydroxycarboxylic acid amides of the above formula (3-2) include, for example,
N-methyl-3-hydroxybutyrylamide,
N-ethyl-3-hydroxybutyrylamide,
N-propyl-3-hydroxybutyrylamide,
N-2-propyl-3-hydroxybutyrylamide,
N-buty-3-hydroxybutyrylamide,
N-2-butyl-3-hydroxybutyrylamide,
N-isobutyl-3-hydroxybutyrylamide,
N-tert-butyl-3-hydroxybutyrylamide,
N-n-pentyl-3-hydroxybutyrylamide,
N-2-pentyl-3-hydroxybutyrylamide,
N-tert-pentyl-3-hydroxybutyrylamide,
N-2-methylbutyl-3-hydroxybutyrylamide,
N-3-methylbutyl-3-hydroxybutyrylamide,
N-2,2-dimethylpropyl-3-hydroxybutyrylamide,
N-n-hexyl-3-hydroxybutyrylamide,
N-2-hexyl-3-hydroxybutyrylamide,
N-3-hexyl-3-hydroxybutyrylamide,
N-tert-hexyl-3-hydroxybutyrylamide,
N-2-methylpentyl-3-hydroxybutyrylamide,
N-3-methylpentyl-3-hydroxybutyrylamide,
N-4-methylpentyl-3-hydroxybutyrylamide,
N-heptyl-3-hydroxybutyrylamide,
N-octyl-3-hydroxybutyrylamide,
N-nonyl-3-hydroxybutyrylamide,
N-decyl-3-hydroxybutyrylamide,
N-lauryl-3-hydroxybutyrylamide,
N-stearyl-3-hydroxybutyrylamide,
N-cyclopropyl-3-hydroxybutyrylamide,
N-cyclobutyl-3-hydroxybutyrylamide,
N-cyclopentyl-3-hydroxybutyrylamide,
N-cyclohexyl-3-hydroxybutyrylamide,
N-phenyl-3-hydroxybutyrylamide,
N-naphthyl-3-hydroxybutyrylamide,
N-anthryl-3-hydroxybutyrylamide,
N-biphenyl-3-hydroxybutyrylamide,
N-benzyl-3-hydroxybutyrylamide,
N-2-phenylethyl-3-hydroxybutyrylamide,
N-1-phenylpropyl-3-hydroxybutyrylamide,
N-3-naphthyl-3-hydroxybutyrylamide,
N-1-phenylethyl-3-hydroxybutyrylamide,
N-2-phenylpropyl-3-hydroxybutyrylamide,
N-3-naphthylpropyl-3-hydroxybutyrylamide, etc.

The optically active 3-hydroxycarboxylic acid derivative represented by the formula (3) includes a (R)-3-hydroxycarboxylic acid derivative represented by the formula (3A): 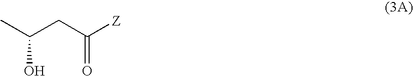
wherein Z has the same meaning as defined above, and a (S)-3-hydroxycarboxylic acid derivative represented by the formula (3B): 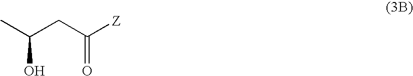
wherein Z has the same meaning as defined above.

In the case where the process of the present invention is carried out using an alcohol as the nucleophilic agent (hereinafter described), the (R)-3-hydroxycarboxylic acid derivative represented by the above formula (3A) can be, for example, a (R)-3-hydroxycarboxylic acid ester represented by the formula (3A-1): 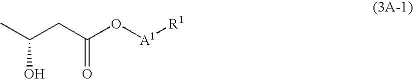
wherein R¹ and A¹ have the same meanings as defined above. Further, in the case where the method of the present invention is carried out using an amine as the nucleophilic agent, the (R)-3-hydroxycarboxylic acid derivative represented by the above formula (3A) can be, for example, a (R)-3-hydroxycarboxylic acid amide represented by the formula (3A-2): 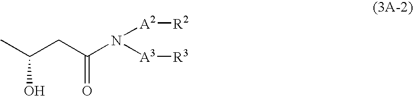
wherein R², R³, A² and A³ have the same meanings as defined above.

In addition, when an alcohol is used as the nucleophilic agent to perform the process of the present invention, the (S)-3-hydroxycarboxylic acid derivative of the formula (3B) can be, for example, a (S)-3-hydroxycarboxylic acid ester represented by the formula (3B-1): 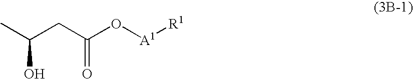
wherein R¹ and A¹ have the same meanings as defined above, and when an amine is used as the nucleophilic agent to perform the process of the present invention, the (S)-3-hydroxycarboxylic acid derivative of the formula (3B) can be, for example, a (S)-3-hydroxycarboxylic acid amide represented by the formula (3B-2): 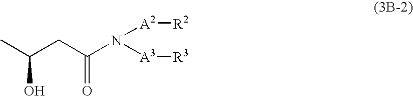
wherein R², R³, A² and A³ have the same meanings as defined above.

In the method of the present invention, the obtained optically active 3-hydroxycarboxylic acid derivative can be preferably a (S)-3-hydroxycarboxylic acid derivative of the above formula (3B), though it depends on the kind of the hydrolase and the nuclophilic agent used, the reaction conditions and the like.

The 3-hydroxycarboxylic acid derivatives obtained by the method of the present invention are produced in an optically active form (optically active 3-hydroxycarboxylic acid derivative) or a racemate, depending on the kind of the hydrolase, the nucleophilic agent, and the like to be used, and the reaction conditions, etc., and preferably an optically active form is produced. Further, the optically active 3-hydroxycarboxylic acid derivatives are also obtained in high optical purity by changing the kind of the hydrolase, the nucleophilic agent, and the like to be used, and the reaction conditions.

The nucleophilic agent used in the present invention includes alcohols, amines, and the like. As such alcohols, they include an alcohol represented, for example, by the formula (4):
R¹-A¹-OH (4)
wherein R¹ and A¹ have the same meanings as defined above.

Specific examples of the alcohol include aliphatic alcohols such as methanol, ethanol, 2-propanol, n-butanol, tert-butanol, pentanol, hexanol, heptanol, octanol, nonanol, decanol, dodecanol and 2-ethoxyethanol; and aromatic alcohols such as benzyl alcohol and phenethyl alcohol, and the like.

As such amines, they include an amine represented, for example, by the formula (5):
R²-A²-NH-A³-R (5)
wherein R², R³, A² and A³ have the same meanings as defined above.

Specific examples of the amines include aliphatic amines such as methylamine, ethylamine, propylamine, butylamine, cyclohexylamine, dimethylamine diethylamine, diisopropylamine, triethylamine, tripropylamine, di(2-ethylhexyl)amine and hexadecylamine; aromatic amines such as benzylamine and phenethylamine; and cyclic amines such as piperidine and morpholine; and the like.

Although there is no particular limitation to the amount of β-butyrolactones as the starting material which are a mixture of optical isomers, and that of the nucleophilic agents, depending on the kind of the nucleophilic agents used, the nucleophilic agent is usually and appropriately used in an amount of 0.1 to 100 mol %, preferably 1 to 50 mol %, to the P-butyrolactones of the starting material.

The hydrolase used in the present invention is a lipase other than lipase derived from porcine pancreas, which includes, for example, carboxyesterase, allyl esterase, choline esterase, lipase, and the like, among which lipase is preferable. In addition, such lipase may be fixed.

Although there is no particular limitation to the lipase so long as it is other than lipase derived from porcine pancreas, there are exemplified lipases belonging to the genus of Candida, Chromobacterium, Pseudomonas, or Geotrichum candidum. Also, lipase derived from Aspergillus niger may be available. Among these lipases, it is preferable to use a lipase derived from the genus Candida.

Examples of the lipases derived from the genus Candida are those derived from Candida antarctica, Candida rugosa, Candida cyndracea or the like. Particularly, lipase derived from Candida antarctica is more preferable, because when such lipase is employed, (S)-β-butyrolactones react with a nucleophilic agent in high selectivity. Furthermore, for example, a commercially available Novozym 435 is preferably employed as a fixed lipase. When the method of the present invention is carried out using an amine as the nucelophilic agent, it is preferable in the present invention to use lipase other than lipase derived from porcine pancreas, though the lipase derived from porcine pancreas may be used.

As to these hydrolases, the commerically available hydrolase may be used as they are. These hydrolases used in the present invention may be used alone or in an appropriate combination with two or more hydrolases thereof.

The amount of the hydrolase used is usually selected from the range of 0.0001- to 2-fold, preferably 0.01- to 1-fold to the β-butyrolactone of starting materials.

Preferable embodiment of the method of the present invention can be carried out by reacting a β-butyrolactone which is a mixture of optical isomers, with a nucleophilic agent in the presence of lipase derived from the genus Candida as the above hydrolase, particularly from Candida antarctica, so that only (S)-β-butyrolactone in the β-butyrolactone which is the mixture of optical isomers reacts substantially to give (S)-3-hydroxycarboxylic acid derivative.

On the other hand, since (R)-β-butyrolactone in the β-butyrolactone which is a mixture of optical isomers does not substantially react with a nucleophilic agent, (R)-β-butyrolactone is obtained in high optical purity. In the above reaction, when an amine is used as the nucleophilic agent, 3-hydroxycarboxylic acid derivative may be produced in the form of substantially a racemate, depending on the kind of the amines used. In order to obtain the (R)-form or (S)-form of 3-hydroxycarboxylic acid derivatives with high optical purity in the above reaction, such (R)- or (S)-form of 3-hydroxycarboxylic acid may be isolated by the hereinafter described post-treatment or purification procedure.

By changing the hydrolase to be used, it is possible to react (R)-β-butyrolactone which is an enantiomer of (S)-β-butyrolactone in the β-butyrolactone which is a mixture of optical isomers as mentioned above, with a nucelophilic agent.

The production method of the present invention, if necessary, may be carried out in the presence of a solvent. Examples of the solvent include hydrocarbons such as aliphatic hydrocarbons such as pentane, hexane, heptane, octane, decane and cyclohexcane, and aromatic hydrocarbons such as benzene, toluene and xylene; halogenated hydrocarbons such as dichloromethane, 1,2-dichloroethane, chloroform, carbon tetrachloride and o-dichlorobenzene; ethers such as diethyl ether, diisoproyl ether, tert-butyl methyl ether, dimethoxyethane, ethylene glycol diethyl ether, tetrahydrofuran, 1,4-dioxane and 1,3-dioxolane; ketones such as acetone, methyl ethyl ketone, methyl isobutyl ketone and cyclohexanone; esters such as methyl acetate, ethyl acetate, n-butyl acetate and methyl propionate; amides such as formamide, N,N-dimethylormamide and N,N-dimethylacetamide; sulfoxides such as dimethyl sulfoxide; organic cyano compounds such as acetonitrile; N-methylpyrrolidone; water; and the like. These solvents may be used alone or in an appropriate combination with two or more solvents thereof.

Although there is no particular limitation to the amount of the solvent to be used, depending on the kind of the nucleophilic agent and the hydrolase to be used and the use amount thereof, it is usually selected from the range of an amount of 0 to 100-fold, preferably 0.1 to 10-fold, to β-butyrolactones which are a mixture of optical isomers of the starting materials.

Although there is no particular limitation to the reaction temperature depending on the kind of the nucleophilic agent or the hydrolase to be used and the use amount thereof, it is usually selected appropriately from the range of 0 to 100° C., preferably 20 to 50° C.

Although there is no particular limitation to the reaction time depending on the kind of the nucleophilic agent or the hydrolase to be used and the use amount thereof, it is usually selected appropriately from the range of 1 to 48 hours, preferably 5 to 24 hours.

Since the (R)-β-butyrolactones and/or (S)-3-hydroxycarboxylic acid derivatives with high optical purity can be prepared by using a hydrolase under mild conditions, there is no necessity to remove by-products and impurities, and post-treatment, if required, gives the desired their compounds in more higher optical purity.

When required, procedures of known methods per se may be used, such as chromatography such as gas chromatography and column chromatography, solvent extraction, pH adjustment, extraction, salting out, crystallization, recrystallization, distillation, and the like. Since the desired compounds can be easily separated and isolated by using these procedures, the objective (R)-β-butyrolactone and/or (S)-3-hydroxycarboxylic acid derivative can be separated in high optical purity.

Thus (R)-β-butyrolactone and/or (S)-3-hydroxycarboxylic acid derivative obtained according to the method of the present invention are useful as intermediates for pharmaceuticals, agrochemicals, and the like.

### EXAMPLES

Hereinafter, the present invention will be illustrated in more detail by way of Examples, but it is to be construed that the present invention is in no way limited to these Examples.

Apparatuses used in the determination of physicochemical properties of compounds are shown below.

Analysis Conditions:

GLC: HP6890(Hewlett Packerd)
HPLC: HITACHI 655LC
   Chemical Purity of β-Butyrolactone and Reaction Products
GC: Neutrabond-1
   Optical Purity of β-Butyrolactone
GC: CHIRALDEX G-TA
   Optical Purity of Hydroxycarboxylic Acid Derivatives
HPLC: CHIRALCEL OD-H

### Example 1

(R)-β-butyrolactone 15.0 g (174 mmol, chemical purity 98.9%, optical purity 92.8% ee), benzyl alcohol 0.72 mL (4 mol %), a fixed lipase derived from Candida antarctica 0.45 g (Product name: Novozym 435) and diisopropyl ether (IPE) 15 mL were placed in a 500 mL-flask, and the mixture was shaken at 35° C. for 16 hours. the optical purity of benzyl (S)-3-hydroxybutanoate resulted in 61.6% ee one hour after the reaction, and that of (R)-β-butyrolactone was 99.7% ee 16 hours after the reaction. After removal of the lipase by filtration, IPE was recovered from the reaction solution, and subjected to crude distillation. As the results, 13.4 g of (R)-β-butyrolactone (chemical purity 99.9%, optical purity 99.6% ee) was obtained in 90.4% yield, and 1.1 g of benzyl (S)-3-hydroxybutanoate (chemical purity 82%) was obtained.

### Example 2

β-butyrolactone 100 g (racemate, chemical purity 98.3%), benzyl alcohol 62.8 g (5 mol %), a fixed lipase derived from Candida antarctica 10 g (Product name: Novozym 435) and IPE 300 mL were placed in a 1L-four necked flask, and the mixture was stirred at 35° C. with a mechanical stirrer for 16 hours. The optical purity of (R)-β-butyrolactone after 16 hours was (99.9% ee. After removal of the lipase by filtration, IPE was recovered from the reaction solution, and then subjected to crude distillation to give 38.88 g of (R)-β-butyrolactone (chemical purity 98.5%, optical purity >99.9% ee was obtained. The yield was 37.7%.

### Example 3

(R)-β-butyrolactone 10.0 g (116 mmol, chemical purity 98.9%, optical purity 92.8% ee), benzylamine 1.27 mL (10 mol %), a fixed lipase derived from Candida antarctica 0.30 g (Product name: Novozym 435) and toluene 10.0 mL were placed in a 500 mL-flask, and the mixture was shaken at 30° C. for 20 hours. After the reaction, the toluene was recovered from the reaction solution, and subjected to crude distillation to obtain 7.47 g of (R)-β-butyrolactone (optical purity 98.4% ee). The yield was 75.6%.

### Example 4

(R)-β-butyrolactone 10.0 g (116 mmol, chemical purity 98.0%, optical purity 90.8% ee), piperidine 1.15 mL (10 mol %), a fixed lipase derived from Candida antarctica 0.30 g (Product name: Novozym 435) and toluene 10.0 mL were placed in a 100 mL-sample bottle, and the mixture was stirred at 30° C. under shaking for 16 hours. After the reaction, the optical purity of (R)-β-butyrolactone was 98.0% ee.

### Example 5

(R)-β-butyrolactone 10.0 g (116 mmol, chemical purity 98.0%, optical purity 90.8% ee), cyclohexylamine 1.33 mL (10 mol %), a fixed lipase derived from Candida antarctica 0.30 g (Product name: Novozym 435) and toluene 10.0 mL were placed in a 100 mL-sample bottle, and the mixture was shaken at 30° C. for 16 hours. After the reaction, the optical purity of (R)-β-butyrolactone was 98.6% ee.

### Example 6

(R)-β-butyrolactone 1.0 g (11.6 mmol, chemical purity 98.9%, optical purity 92.8% ee), octanol 0.18 mL (10 mol %), a fixed lipase derived from Candida antarctica 30 mg (Product name: Novozym 435) and IPE 1.0 mL were placed in a 10 mL-sample bottle, and the mixture was shaken at 35° C. for 16 hours. After 17 hours, the optical purity of (R)-β-butyrolactone was 99.3% ee, and that of octyl (S)-3-hydroxybutanoate was 8.5% GC.

### Example 7

(R)-β-butyrolactone 1.0 g (11.6 mmol, chemical purity 98.9%, optical purity 92.8% ee), benzyl alcohol 0.12 mL (10 mol %), a fixed lipase derived from Candida antarctica 0.1 g (Product name: Chirazyme L2) and IPE 1.0 mL were placed in a 10 mL-sample bottle, and the mixture was shaked at 35° C. for 16 hours. The optical purity of (R)-β-butyrolactone was 99.4% ee and that of benzyl (S)-3-hydroxybutanoate was 63.1% ee, after 16 hours.

### INDUSTRIAL APPLICABILITY

The present invention provides optically active β-butyrolactones and/or optically active 3-hydroxycarboxylic acid derivatives in high optical purity, which are useful as intermediates for pharmaceuticals, agrochemicals and the like.

1. A method for preparing an optically active β-butyrolactone, which comprises reacting a β-butyrolactone which is a mixture of optical isomers, with a nucleophilic agent in the presence of a hydrolase, provided that a lipase derived from porcine pancreas is excluded.

2. The method according to claim 1, wherein the optically active β-butyrolactone obtained is (R)-p-butyrolactone.

3. The method according to claim 1, wherein the β-butyrolactone which is a mixture of optical isomers is a β-butyrolactone with low optical purity, and the β-butyrolactone obtained is a β-butyrolactone with high optical purity.

4. The method according to claim 1, wherein the optical purity of the β-butyrolactone obtained is substantially 100% ee.

5. A method for preparing an optically active β-butyrolactone and/or an optically active 3-hydroxycarboxylic acid derivative, which comprises reacting a butyrolactone which is a mixture of optical isomers, with a nucleophilic agent in the presence of a hydrolase, provided that a lipase derived from porcine pancreas is excluded.

6. The method according to claim 5, wherein the optically active α-butyrolactone obtained and/or the optically active 3-hydroxycarboxylic acid derivative obtained is/are isolated.

7. The method according to claim 5, wherein the 3-hydroxycarboxylic acid derivative obtained is an optically active 3-hydroxycarboxylic acid derivative.

8. The method according to claim 5, wherein the optically active β-butyrolactone obtained is an (R)-butyrolactone, and the optically active 3-hydroxycarboxylic acid derivative obtained is an (S)-3-hydroxycarboxylic acid derivative.

9. The method according to claim 5, wherein the β-butyrolactone which is a mixture of optical isomers is a β-butyrolactone with low optical purity, and the β-butyrolactone obtained is a β-butyrolactone with high optical purity.

10. The method according to claim 5, wherein the optical purity of the (R)-β-butyrolactone obtained is substantially 100% ee.

11. The method according to claim 1, wherein the hydrolase is a lipase, provided that a lipase derived from porcine pancreas is excluded.

12. The method according to claim 11, wherein the lipase is a lipase belonging to the genus Candida.

13. The method according to claim 12, wherein the lipase belonging to the genus Candida is a lipase derived from Candida antarctica.

14. The method according to claim 1, wherein the nucleophilic agent is an alcohol or an amine.

15. The method according to claim 5, wherein the hydrolase is a lipase, provided that a lipase derived from porcine pancreas is excluded.

16. The method according to claim 15, wherein the lipase is a lipase belonging to the genus Candida.

17. The method according to claim 16, wherein the lipase belonging to the genus Candida is a lipase derived from Candida antarctica.

18. The method according to claim 14, wherein the nucleophilic agent is an alcohol or an amine.
